(19) United States
(12) Patent Application Publication
(10) Pub. No.: US 2016/0157728 A1
(43) Pub. Date: Jun. 9, 2016

(54) ACOUSTIC-ELECTRIC IMAGING METHOD AND DEVICE

(71) Applicant: CENTRE NATIONAL DE LA RECHERCHE SCIENTIFIQUE-CNRS-, Paris Celldex 16, (FR)

(72) Inventors: Mickaël Tanter, Bagneux (FR); Mathieu Pernot, Paris (FR); Mathias Fink, Meudon (FR); Jean Provost, Paris (FR)

(21) Appl. No.: 14/907,178

(22) PCT Filed: Jul. 21, 2014
(86) PCT No.: PCT/FR2014/051880

§ 371 Date: Jan. 22, 2016

(30) Foreign Application Priority Data
Jul. 22, 2013 (FR) 13 57178

(51) Int. Cl. A61B 5/00

(52) U.S. Cl. CPC A61B 5/0093; A61B 2576/00

(57) ABSTRACT

The invention relates to an acoustic-electric imaging method, which includes: a measurement step during which incident ultrasonic waves having different wavefronts are emitted in a medium 1 to be imaged, and at least one electric sensor is used to capture raw electric signals Erawl(t) respectively during the propagation of the incident waves; a step of forming an image, during which an image of the medium including an electric current map is determined from the raw electric signals Erawl(t).

### FIELD OF THE INVENTION

The present invention relates to methods and devices for acoustic-electric imaging.

Organs such as the heart, the skeletal muscles, and the brain are continuously traveled by electrical impulses that carry the information in the neurons, or that trigger muscle or myocardial contraction. It is extremely important to be able to image the propagation of these impulses in order to diagnose many diseases and to understand brain mechanisms through functional exploration of the brain.

Acoustic-electric imaging exploits the interaction between ultrasound and electric currents to determine the value of the electric current at points of interaction between ultrasound and tissue, typically at the focal spot of a focused ultrasonic wave.

### BACKGROUND OF THE INVENTION

U.S. Pat. No. 8,057,390 discloses an example of an acoustic-electric imaging method, in which focused ultrasonic waves are emitted so as to form an image of the current, line by line. This acquisition process is slow, and all the more so because, as the resulting electrical signals are very weak, a high level of averaging is required. Low frame rates are therefore obtained.

Kuchment et al., in “Synthetic focusing in ultrasound modulated tomography,” Inverse problem and imaging, 2009-10-01, pages 1-9, XP055116447, proposed a method for synthetic acoustic-electric imaging, in which the transducers emit spherical waves, one by one. The result is a slow process. In addition, the incident ultrasonic waves have too low of an amplitude.

### SUMMARY OF THE INVENTION

The present invention is intended to overcome this disadvantage.

To this end, the invention proposes a method for acoustic-electric imaging, comprising:

(a) a measurement step during which an array of transducers Tᵢ emits, in a field of view of a medium to be imaged, a number N at least equal to 2 of incident ultrasonic waves l that are not focused in the field of view and that have different wavefronts, each incident ultrasonic wave being emitted by a plurality of transducers Tᵢ among the array of transducers, where N is at least equal to 2 and less than 100, and at least one electric sensor, in contact with the medium to be imaged, captures raw electrical signals Erawₗ(t) respectively during the propagation of the incident waves l,

(b) an image formation step, during which an image of the medium comprising a map of the electric currents (in other words, a map of the electrical values representative of local current densities at each point of the medium) is determined from the raw electrical signals Erawₗ(t) obtained in step (a).

With these arrangements, one can obtain ultrafast imaging of electrical impulses in the medium observed, and possibly film the propagation of electrical impulses deep in the tissue in real time and at a millimeter resolution.

In various embodiments of the method according to the invention, one or more of the following arrangements may possibly be used:
during step b), at least from the N raw electrical signals Erawₗ(t), for a number M of fictitious focal points Pₖ in the field of view, electrical values Ecoherentₖ are determined, each corresponding to the electrical signal that would have been captured if an ultrasonic wave focused at point Pₖ had been emitted by said transducers;
during step (b), an inverse wavelet transform WT⁻¹ and then an inverse Radon transform R⁻¹ are applied to the raw electrical signals Erawₗ(t) (the raw electrical signals Erawₗ(t) may of course undergo preliminary processing prior to the inverse Radon transform R⁻¹);
during step (b), an ultrasound image of the medium, created with the transducer array, is superimposed on the map of electric currents;
during step (a), the transducers Tᵢ capture acoustic signals RFraw_{l,i}(t) representative of ultrasonic waves reverberated by the medium respectively from the incident waves l,
during step (b), from the N sets of captured signals RFraw_{l,i}(t), M coherent acoustic signals RFcoherent_{k,i}(t) are determined which correspond to the acoustic signals that would have been received by the transducers Tᵢ if an ultrasonic wave focused at point Pₖ had been emitted by said transducers, and the ultrasound image of the medium is calculated from the coherent acoustic signals;
during step (b), the ultrasound image is determined by beamforming based on the coherent acoustic signals;
the medium to be imaged is human or animal tissue.

The invention also relates to a device for implementing a method for acoustic-electric imaging, comprising an array of transducers Tᵢ, least one electric sensor, and control and processing means adapted for:

(a) causing an array of transducers Tᵢ to emit, in a medium to be imaged, a number N of unfocused incident ultrasonic waves l having different wavefronts, each incident ultrasonic wave being emitted by a plurality of transducers Tᵢ among the array of transducers, N being at least equal to 2 and less than 100, and causing at least one electric sensor, in contact with the medium to be imaged, to capture raw electrical signals Erawₗ(t) respectively during the propagation of the incident waves l,

(b) determining an image of the medium, comprising a map of the electric currents, from the raw electrical signals Erawₗ(t).

### BRIEF DESCRIPTION OF DRAWINGS

Other features and advantages of the invention will be apparent from the following description of one of its embodiments, given by way of non-limiting example and with reference to the accompanying drawings.

In the drawings:

FIG. 1 is a schematic view of a device for implementing a method according to an embodiment of the invention, and

FIG. 2 is a block diagram of a portion of the device of FIG. 1.

### DETAILED DESCRIPTION

In the various figures, the same references designate identical or similar elements.

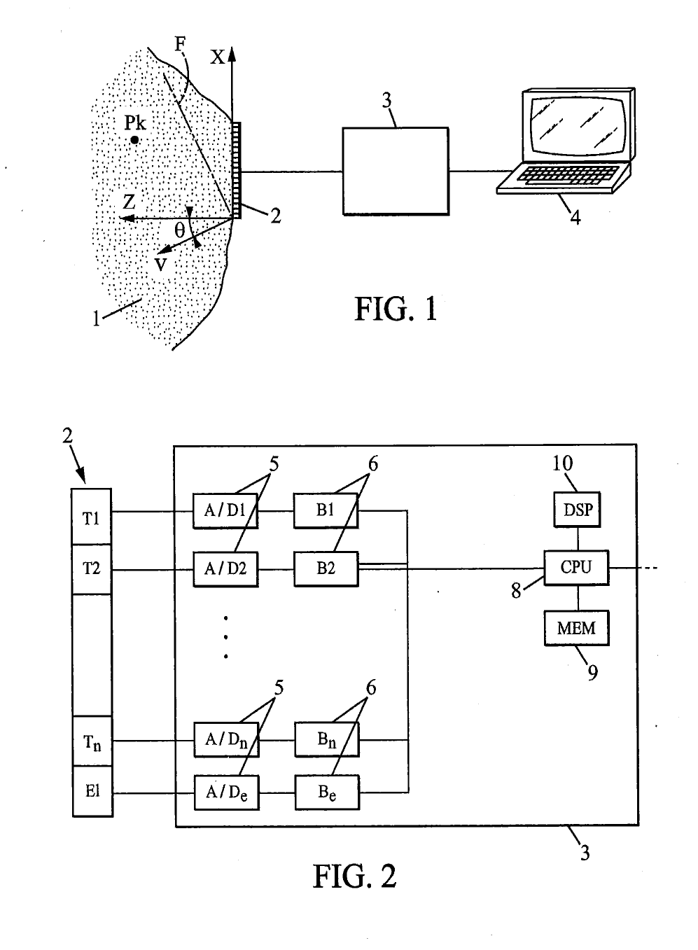

FIG. 1 shows an example of an acoustic-electric imaging device adapted for imaging a medium 1 by emitting and receiving ultrasonic compression waves (for example at frequencies between 0.2 and 40 MHz), with simultaneous measurement of electrical values.

The medium 1 to be imaged may consist in particular of tissues of a patient or an animal, in particular muscle (myocardium or other) or brain.

The imaging device comprises, for example:
an array 2 of n ultrasonic transducers, comprising for example several hundred transducers and adapted for obtaining a two-dimensional (2D) image of a field of view (area of interest scanned by the ultrasonic waves) in the medium 1 to be imaged;
an electronics bay 3 or the like for controlling the transducer array 2 and adapted for acquiring the signals captured by the transducer array;

a computer 4 or the like for controlling the electronics bay 3 and for viewing ultrasound images obtained from said captured signals.

The transducer array 2 may, for example, be a linear array formed by a set of transducers placed next to one another along an axis X, with the Z axis perpendicular to the X axis denoting the depth direction in the field of view. In what follows, the transducers will be denoted Tᵢ, where i is an index indicating the position of each transducer along the axis X. The following description uses this type of transducer array 2 for its example, but other forms of transducer array are also possible within the scope of the invention, including two-dimensional arrays.

The device further comprises at least one electric sensor El (FIG. 2), formed for example by two electrodes measuring a difference in electric potential. This electric sensor may advantageously be attached to the transducer array 2 and be adapted to come into contact with the medium 1 to be imaged at the same time as the transducer array 2.

The number of electric sensors El used is relatively low, generally less than 10, preferably less than 5, and usually 1.

As represented in FIG. 2, the electronics bay 3 may comprise for example:
n+1 analog-to-digital converters 5 (A/Dᵢ-A/Dₑ) individually connected to the n transducers Tᵢ in the transducer array 2 and to the electric sensor El,
n+1 buffers 6 (Bᵢ-Bₑ) respectively connected to the n analog-to-digital converters 5,
a central processing unit 8 (CPU) communicating with the buffers 6 and the computer 4,
a memory 9 (MEM) connected to the central processing unit 8,
a digital signal processor 10 (DSP) connected to the central processing unit 8.

Note that the n+1 analog-to-digital converters 5 (A/Dᵢ-A/Dₑ) may be identical, which is also the case for the n+1 buffers 6 (Bᵢ-Bₑ), so that the device used may simply be a device as conventionally used in ultrafast acoustic imaging.

This device allows implementing a method of acoustic-electric imaging of the medium 1, which in particular includes the following steps, carried out by the central processing unit 8 assisted by the processor 8 and the digital signal processor 10:
a) measuring (transmission/reception and saving of raw data),
b) determining an image of the medium comprising a map of electrical values.

### Step (a): Measuring (Transmission/Reception and Saving of Raw Data):

The transducer array 2 and the electric sensor El are placed in contact with the medium 1 and a number N of incident ultrasonic waves is emitted into medium 1 by the transducers Tᵢ (N may be for example between 2 and 100, in particular between 5 and 10). The incident waves in question are unfocused (more specifically, not focused in the field of view) and have different respective wavefronts, meaning wavefronts of different shapes and/or different orientation. Advantageously, the incident waves are plane or divergent waves whose respective wavefronts F (the wavefront F of a single wave is represented in FIG. 1) have various different inclinations, characterized by their respective angles of inclination θ measured between their direction of propagation V and the Z axis, or divergent waves emitted as if they originated from different points in space. The example of plane waves will be considered in the following.

The incident waves are generally pulses of less than a microsecond, typically about 1 to 10 cycles of the ultrasonic wave at the center frequency. The firing of incident waves may be space apart, for example by about 50 to 200 microseconds.

Each incident wave encounters reflectors in the medium 1, which reverberate the incident wave. The reverberated ultrasonic wave is captured by the transducers Tᵢ of the array. The signal thus captured by each transducer Tᵢ comes from the medium 1 as a whole, since the incident wave is not focused at emission. Similarly, the electric sensor El captures an electrical signal E(t) during propagation of the incident ultrasonic wave, and this electrical signal results from the interaction between the incident wave and the medium 1 to be imaged, along the entire line represented by the wavefront, at each measurement time.

Reverberant signals captured by the n transducers Tᵢ are then digitized by the corresponding analog-to-digital converters A/Dᵢ and stored in the corresponding buffers Bᵢ, while the electrical signal is digitized by the analog-to-digital converter A/Dₑ and stored in the corresponding buffer Bₑ. These signals stored in the buffers after each incident firing will be referred to hereinafter as raw data. These raw data consist of n+1 raw time signals RFraw_{l,i}(t) and Erawₗ(t) respectively captured by the transducers Tᵢ and the electric sensor El after the firing l of incident ultrasonic waves.

After each firing l of incident waves, the signals stored in the buffers Bi-Bₑ are transferred to the memory 9 of the signal processor 10 for processing by said processor. At the end of step (a), the memory 9 therefore contains N arrays (vectors) of n+1 raw signals.

Step (a) is repeated at a fast rate, such as 500 Hz or more, which is made possible by the low number N of incident waves used to obtain an image.

### Step (b): Determining an Image of the Medium Comprising a Map of Electrical Values:

Two methods will be explained below for carrying out this step (b).

b1) First Method: Synthesis of Coherent Data:

From N arrays of raw data, a number M of arrays (vectors) of synthetic coherent data is calculated by the processor 8, respectively at M points Pₖ(x,z) of the field of view (k being an integer between 1 and M, and x, z being the coordinates of point Pₖ on the X, Z axes). Each of these M vectors of synthetic coherent data contains n time signals RFcoherent_{k,i}(t) corresponding to the signals which would respectively be captured by the transducers Tᵢ if the transducers were emitting an incident wave focused at point Pₖ.

The arrays of coherent data may be obtained for example by assuming a uniform propagation speed c for ultrasonic compression waves throughout the medium 1, according to the principle explained in particular in document EP2101191 or in the article by Montaldo et al.: “Coherent plane-wave compounding for very high frame rate ultrasonography and transient elastography” (IEEE Trans Ultrasound Ferroelectr Freq Control 2009 March; 56(3): 489-506).

As the direction of propagation of the plane wave corresponding to the each firing l is known, and the propagation speed v is known, the processor 8 can calculate for each point Pₖ the propagation time τ_{ec}(l,k) of the incident wave l to point Pₖ, and the propagation time τ_{rec}(l,k,i) of the reverberated wave from point Pₖ to the transducer Tᵢ, therefore the total round trip travel time τ(l,k,i)=τ_{ec}(l,Pₖ)+(l,Pₖ,i).

The spatially coherent acoustic signal for transducer Ti, corresponding to virtual focal point Pₖ, is then calculated using the formula:

$${RFcoherent}_{kij} = {\sum }_{l} B ( l ) {RFraw}_{lij} ( τ ( l , k , i , j ) ) \qquad ( 1 )$$

where B(l) is a weighting function for the contribution of each firing l of incident waves (in the current cases, the values of B(l) may all be equal to 1). This signal RFcoherentₖᵢⱼ presents a single value for each point Pₖ.

In the same manner, one can calculate a coherent electrical signal Ecoherentₖ:

$${Ecoherent}_{k} ( t ) = {\sum }_{l} B ( l ) {Eraw}_{l} ( τ ( l , k , i , j ) ) \qquad ( 1 a )$$

This electrical value is the one that would be measured by the electric sensor El if an incident ultrasonic wave focused at Pₖ had been emitted, particularly if a sufficient number of incident waves are emitted to obtain an acoustic-electric image, for example 40 to 100 incident waves to obtain a high-resolution image.

These values Ecoherentₖ are representative of the electric currents at the points Pₖ, in the same manner as the electrical values captured in the known acoustic-electric imaging methods mentioned above, and therefore provide a map of the electric currents within the field of view.

The arrays of coherent data RFcoherentₖ and possibly the values Ecoherentₖ may then possibly be refined by correcting the effects of aberrations in the medium 1, for example as described for example in patent EP2101191 or in the document by Montaldo et al: “Coherent plane-wave compounding for very high frame rate ultrasonography and transient elastography” (IEEE Trans Ultrasound Ferroelectr Freq Control 2009 March; 56(3): 489-506).

The electric current map can be presented on the screen of the computer 4, possibly superimposed on a B-mode ultrasound image of the medium 1 or on some other image of said medium 1, in particular an ultrasound image obtained from the arrays Ecoherentₖ by beamforming in receive mode, for example as explained in said document EP2101191.

b2) Second Method: Radon and Wavelet Transform:

From the electrical signals Erawₖ(t), it is also possible to send directly the local values of electric currents at the points Pₖ, as will be explained below.

The raw electrical signal Erawₖ(t) can be modeled as follows:

Erawₖ=∫ᵥₒₗᵤₘₑKρJ(x,y,z)ΔP(x,y,z)dxdydz (2)

where:
K is an interaction constant on the order of 10⁻⁹ Pa⁻¹,
ρ is the resistivity of the medium,
ΔP is the pressure variation,
y is a coordinate along a Y axis perpendicular to plane (X, Z), and
J is the density distribution of the detected current, in other words the scalar product of the current density vector times the electrode sensitivity vector of the electrodes of the electric sensor El.

As the ultrasonic wave emitted is a pulse plane wave, ΔP(x,y,z) can be configured as a function of the emission angle θ and the time t. By ignoring the direction Y, we have:

ΔP(x,z)=ΔP(−q sin θ+ct cos θ,q cos θ+ct sin θ),

where q and ct are coordinates respectively in the direction of the wave front F and in the direction of propagation V.

By considering the emitted ultrasonic wave as a Dirac impulse, meaning an infinitely short impulse, the acoustic-electrical signal becomes:

$$\frac{{Eraw}_{k}}{K ρ} = {\int }_{- ∞}^{+ ∞} J ( - q \cdot sin ϑ + ct \cdot cos θ , q \cdot cos θ + ct \cdot sin θ ) d q \qquad ( 3 )$$

or equivalently:

$$\frac{{Eraw}_{k}}{K ρ} = RJ ( θ , ct ) = {\int }_{- ∞}^{+ ∞} {\int }_{- ∞}^{+ ∞} J ( x , z ) \cdot δ ( x \cdot sin θ + z \cdot cos θ - ct ) d x d z \qquad ( 4 )$$

where R[J] is the Radon transform.

In practice, the incident wave is not a Dirac impulse but an impulse signal of finite frequency band, which will result in a convolution relative to variable ct of the Radon transform:

$$\frac{{Eraw}_{k}}{K ρ} ( θ , t ) = W ( ct ) ⊗ RJ ( θ , ct ) \qquad ( 5 )$$

where W(ct) is the waveform emitted and \textcircled{x} is the convolution product.

For example, a typical ultrasound emission produces the following convolution kernel:

$$W ( ct ) = {e}^{\frac{- {( ct )}^{2}}{- {( n λ )}^{2}}} sin \frac{ct}{m λ} \qquad ( 6 )$$

where n and m can be adjusted within the frequency band of the transducer. This convolution kernel is equivalent to a ridgelet transform [E. J. Candes, “Ridgelets: theory and applications,” Stanford University, 1998] of the current density distribution.

In practice, m=n and this convolution kernel becomes a ridgelet decomposition with the following parameters: a=nλ, b=ct and θ.

The ridgelet decomposition has several mathematical properties, such as a Parseval-Plancherel relation, a reconstruction formula, a sparse representation of slowly varying objects far from linear discontinuities, and can be expressed as a composition of a wavelet transform and the Radon transform.

More specifically, by denoting the wavelet and ridgelet transforms as WT[.] and RT[.] respectively, it can be demonstrated that

$$\frac{{Eraw}_{k}}{K ρ} = WT [ R ( J ) ] \qquad ( 7 )$$

Inversions of the wavelet transform and Radon transform are well-known problems. Exact inversions exist, respectively WT⁻¹ and R⁻¹ for these two transforms, and we therefore have:

$$J = {R}^{- 1} [ {WT}^{- 1} ( \frac{{Eraw}_{k}}{K ρ} ) ] . \qquad ( 8 )$$

In practice, the inversion occurs in two steps: first, inverting the wavelet transform WT, then inverting the Radon transform R.

This provides a current density map across the entire field of view (area swept by the incident waves) within the medium to be imaged, and does so after very fast acquisition, allowing real-time monitoring of very rapid electrical phenomena by obtaining an actual movie of the propagation of electrical impulses.

It is also desirable to maximize the signal-to-noise ratio (SNR), the resolution, and the frame rate.

One approach is to emit the incident waves in the form of the shortest possible impulses, thereby optimizing the resolution. However, this corresponds to emitting very low energy and therefore a low SNR.

It is also possible to divide the frequency band into sub-bands corresponding to longer emissions (and therefore more energy). In theory, there is a resulting increase in the SNR, but with a decrease in the frame rate (because it takes multiple emissions to form an image).

Lastly, a third approach involves emitting a “chirp” which can be used to perform the impulse compression. This approach maximizes the SNR while maintaining the frame rate.

The SNR can also be improved by limiting the effect of noise. Because the ridgelet transform is a sparse basis which represents the current density distribution with a small number of large coefficients and a large number of small coefficients, noise can be eliminated simply by applying a threshold to the signals obtained. A first approach consists of thresholding to eliminate the ‘small’ coefficients. Otherwise, it is also possible to use the physics of the problem. For example, the coefficients primarily containing noise can be identified by cross-correlation between windows of signals received for two emissions of opposite polarities. In addition, these signals can be subtracted to eliminate systemic artifacts.

Several techniques exist for inversion of the Radon transform. The most common is probably filtered back projection, which involves the application of a ramp filter before the back projection (corresponding to the beamforming). To avoid this step, which increases the noise level, it is also possible to emit the incident ultrasonic waves in the form of a suitable impulse which includes this filter. Other strategies such as compressed sensing are also suitable.

In addition, the arrays RFcoherentₖ can be calculated as explained above in method b1), in order to further form a two-dimensional (B-mode) ultrasound image of the field of view by beamforming in receive mode, as explained for example in said document EP2101191.

This B-mode ultrasound image (or some other image, possibly ultrasound) of the field of view may possibly be superimposed on the previously determined map of electrical values, and both the ultrasound image of the medium and the electric current map can be displayed on the computer 4 screen.

1. A method for acoustic-electric imaging, comprising:
a measurement step during which an array of transducers Ti emits, in a field of view of a medium to be imaged, a number N at least equal to 2 of incident ultrasonic waves 1 that are not focused in the field of view and that have different wavefronts, each incident ultrasonic wave being emitted by a plurality of transducers Ti among the array of transducers, N being at least equal to 2 and less than 100, and at least one electric sensor, in contact with the medium to be imaged, captures raw electrical signals Erawl(t) respectively during the propagation of the incident waves 1,
an image formation step, during which an image of the medium, comprising a map of the electric currents, is determined from the raw electrical signals Erawl(t) obtained in step.

2. The method according to claim 1, wherein, during step b), at least from the N raw electrical signals Erawl(t), for a number M of fictitious focal points Pk in the field of view, electrical values Ecoherentk are determined, each corresponding to the electrical signal that would have been captured if an ultrasonic wave focused at point Pk had been emitted by said transducers.

3. The method according to claim 1, wherein, during step, an inverse wavelet transform WT-1 then an inverse Radon transform R-1 are applied to the raw electrical signals Erawl(t).

4. The method according to claim 1, wherein, during step, an ultrasound image of the medium, obtained with the array of transducers, is superimposed on the map of electric currents.

5. The method according to claim 4, wherein, during step, the transducers Ti capture acoustic signals RFrawl,i(t) representative of ultrasonic waves reverberated by the medium respectively from the incident waves 1,
during step, from the N sets of captured signals RFrawl,i(t), M coherent acoustic signals RFcoherentk,i(t) are determined which correspond to the acoustic signals that would have been received by the transducers Ti if an ultrasonic wave focused at point Pk had been emitted by said transducers, and the ultrasound image of the medium is calculated from the coherent acoustic signals.

6. The method according to claim 5, wherein, during step, the ultrasound image is determined by beamforming based on the coherent acoustic signals.

7. The method according to claim 1, wherein the medium to be imaged is human or animal tissue.

8. A device for implementing a method for acoustic-electric imaging according to any claim 1, comprising an array of transducers Ti, at least one electric sensor, and control and processing means adapted for:
causing the array of transducers Ti to emit, in a medium to be imaged, a number N of unfocused incident ultrasonic waves 1 having different wavefronts, each incident ultrasonic wave being emitted by a plurality of transducers Ti among the array of transducers, N being at least equal to 2 and less than 100, and causing at least one electric sensor, in contact with the medium to be imaged, to capture raw electrical signals Erawl(t) respectively during the propagation of the incident waves 1,
determining an image of the medium, comprising a map of the electric currents, from the raw electrical signals Erawl(t).
